(19) United States
(12) Patent Application Publication
(10) Pub. No.: US 2007/0238112 A1
(43) Pub. Date: Oct. 11, 2007

(54) Microfluidic and nanofluidic electronic devices for detecting changes in capacitance of fluids and methods of using

(75) Inventors: Lydia Lee Sohn, Oakland, CA (US); Omar A. Saleh, Holmdel, NJ (US); James Bradford Knight, Glencoe, IL (US); Daniel A. Notterman, Princeton, NJ (US); Laura F. Landweber, Princeton, NJ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Trustees of Princeton University, Princeton, NJ (US)

(21) Appl. No.: 11/586,337

(22) Filed: Oct. 24, 2006

Related U.S. Application Data
(63) Continuation of application No. 10/069,809, filed on Nov. 13, 2002, now abandoned.
(60) Provisional application No. 60/150,899, filed on Aug. 26, 1999.
(60) Provisional application No. 60/211,063, filed on Jun. 13, 2000.

(51) Int. Cl. C12Q 1/68; C12M 1/34

(52) U.S. Cl. 435/6; 435/287.1

(57) ABSTRACT

The present invention (also identified as “Capacitance cytometry”) relates to microfluidic and nanofluidic devices for detecting or measuring an electrical property of a fluid (liquid or aerosol), a single molecule, particle, or cell in fluid. In a particular embodiment, the devices detect or measure changes in capacitance of a fluid, molecule, particle or cell as it passes through the device. The invention relates to detection and measurement of single molecules, particularly biological molecules, and to methods of sequencing polynucleotide molecules (RNA or DNA) by detecting differentially labeled single nucleotides. Single molecule detection applications include DNA or RNA sequencing, detection of SNPs, protoemics, and particle sizing. The device can be used to determine cell DNA content, to analyze cell-cycle kinetics of cell populations, and to assay for abnormal changes in cell DNA content. Nano-microfluidic devices of this invention also have utility as detectors in molecular sorting systems and for detecting pathogens.

### RELATED APPLICATIONS

This application claims priority to U.S. Provisional Application No. 60/150,899 filed Aug. 26, 1999, and U.S. Provisional Application No. 60/211063 entitled “Single Molecule Sequencing” filed Jun. 12, 2000, each of which are incorporated herein by reference in their entirety.

### GOVERNMENT SUPPORT

This invention was made with U.S. governmental support under NSF Grant No. DMR 96-24536. The U.S. government has certain rights in the invention.

### FIELD OF THE INVENTION

The present invention relates to microfluidic and nanofluidic electrical devices for detecting or measuring an electrical property of a fluid including a liquid or aerosol, a single molecule, or a single particle or cell in a fluid. In a particular embodiment, the devices detect or measure changes in capacitance of a fluid, gas, molecule, particle or cell as it passes through the device. The present invention also relates to the detection and measurement of single molecules, in particular, biological molecules. The present invention also relates to methods of sequencing polynucleotide molecules, such as RNA or DNA, by detecting differentially labeled single molecules. The present invention relates to methods of detecting cells in a fluid and methods of measuring cellular components such as, but not limited to, DNA, RNA or protein. In a particular embodiment, the microfluidic device is capable of measuring the DNA content of individual cells in a fluid. The invention also relates to methods of analyzing the cell-cycle kinetics in a population of cells using the microfluidic devices of the invention. Further, the invention relates to methods of detecting malignant cells from a population of cells. The invention also relates methods of detecting environmental monitoring of fluids, including liquids or aerosols, for the presence of pathogens, spores etc. Lastly, the present invention relates to methods of medical diagnosis where changes in DNA content of cells or changes in the cell-cycle kinetics of a population of cells is indicative of a disease state.

### BACKGROUND OF THE INVENTION

A variety of methods exist for measuring properties of fluids, gases and biological samples in fluids. The ability to record the response of a small amount of fluid or a single biomolecule to a nanoscale or microscale probe can be the basis for a powerful new class of molecular-sensing devices. However, the realization of such a probe is extremely challenging given that the necessary technologies for single-molecule detection, isolation, and identification are just emerging and given that the fabrication technology needed to access the length scales compatible with single biomolecules (˜10 nm) is still very much in development. A host of applications, including rapid DNA sequencing, protein identification, and ultrasensitive chemical analysis, would become possible were these enabling technologies fully developed.

Previously, strategies to detect single molecules has primarily focused on a select set of optical techniques such as laser-induced fluorescence, confocal and near-field microscopies, and optical trapping. While each of these has proved successful in specific applications, a number of limitations, including photobleaching and photodamage, restrict their general application to sensors. This prompts the question as to whether any other technique is viable. In this regard, electronic detecting schemes which make use of state-of-the-art nanofabrication and microfabrication technology and nanoelectronics and microelectronics may provide the answer as they represent a significant advance over optical methods with respect to sample damage, size and parallel throughput.

For example, sequencing of DNA currently is a labor intensive process which involves cloning, chemical or enzymatic manipulation, and sample analysis on capillary or vertical gels. More recently, higher through-put DNA sequencing utilizes fluorescent probes to detect the chemically modified DNA molecules.

While the process of DNA sequencing has been made more efficient through sequencing machines (for example from the manufacturerers, ABI, Licor, Pharmacia, etc.) and mass-spectrometry, the process is still brute force, requiring amplification and manipulation of the DNA. The processes still remain costly at approximately five or more cents per base pair. Therefore, sensors that enable direct, non-enzymatic rapid DNA sequencing would revolutionize biology, as this has the potential to increase DNA sequencing throughput by many orders of magnitude.

Likewise, protein are routinely examined by using polyacrylamide gels that can separate proteins based on their size and/or their overall charge. The proteins separated on the gel are either stained nonspecifically or detected with antibodies that bind to specific proteins. The non-specific stain is not extremely accurate, as many proteins can have similar sizes or charges. The process of separating proteins in standard gel is also labor-intensive. Furthermore, purifying a protein and generating antibodies specific to that protein take several months under the best conditions. An additional method for identifying proteins involves mass-spectrometry of purified proteins, but this is difficult and time consuming, as the proteins must be individually purified. Therefore, rapid and unique identification of single proteins would have a great impact on the pharmacological industry and field of medicine for disease detection and treatment.

A number of chemical and biosensors are available. For example, a capacitive chemical sensor is described in U.S. Pat. No. 4, 822,566 which detects an analyte in a solution but requires biospecific binding between a biochemical binding system and the analyte.

Another sensor for molecular structures is described in U.S. Pat. No. 5,846,708 issued to Hollis et al. which electrically senses the binding of a molecule to a test site by hybridization.

Another commonly used method, flow cytometry, is a technique for rapid measurement of biological and physical properties of cells and particles. It involves analysis of directly scattered or Storkes-shift light (fluorescence) from cells in a fast-flowing fluid stream, illuminated by a strong light source, usually a laser, and referred to as laser flow cytometry. Information concerning physical properties of the cells including shape and size may be derived from the directly scattered light. Cells may be labeled with fluorescent probes to determine biological properties such as DNA, RNA and protein content. A variety of properties may be studied simultaneously using multiple wavelength excitation. (See, for example, “Pulsed Laser Flow Cytometry, WO 92/08120 and U.S. Pat. No. 5,041,733 issued to Noguchi et al., Aug. 20, 1991). For example, Coulter counters currently in use, measure particles or cells in a fluid by measuring the displaced volume as cells or particles flow through a small orifice (A. Yen (1989) in Flow Cytometry: Advanced Research and Clinical Applications, (CRC Press)).

Although standard laser flow cytometry is also able to analyze individual cells, it requires sample preparation such as sample staining or manipulation of cells. Such sample preparation often destroys the sample. There is a need for a way to analyze individual cells without time consuming, costly, and destructive special sample preparation.

In the past, capacitance measurements have been used to identify and investigate a number of different materials in bulk (Pethig, Ronald (1979) in Dielectric and Electronic Properties of Biological Materials, (John Wiley and Sons, Ltd) pp. 1-376). More recently, it has been used to investigate ensembles of biological cells for determining cell size and cellular membrane capacitance in order to assay cell-cycle progression (Asami, K., Gheorghiu, E. & Yonezawa, T. (1999) Biophysical Journal 76, 3345-33484) and to differentiate normal and malignant white blood cells (Polevaya, Y., Ermolina, I., Schlesinger, M., Ginzburg, B. Z. & Feldman, Y. (1999) Biochim. Biophys. Acta 15, 257-715). However, there is a need for a device capable of employing capacitance measurements as a means of detecting and quantifying the polarization response of DNA within the nucleus of single eukaryotic cells.

The electrical properties or characteristics of biological cells are of great interest, as they can provide opportunities to develop novel, rapid assays for disease (See, for example, Ayliffe, H. E., Frazier, A. B. & Rabbitt, R. D. (1999) IEEE Journal of Microelectro-mechanical Systems 8, 50-57; and Huang Y., Yang J., Wang X. B., Becker F. F. & Gascoyne P. R. C. (2000) Journal of Hematotherapy and Stem Cell Research 8, 481-490) and integrated hybrid chips for electronics (See, for example, Fromherz, P., Kiessling, V., Kottig, K. & Zeck, G. (1999) Applied Physics A 69, 571-576; Vassanelli, S. & Fromherz, P. ( 1997) Applied Physics A 65, 85-88; and Maher, M. P., Pine, J., Wright, J. & Yu-Chong Tai. (1999) Journal of Neuroscience Methods 87, 45-56; Kawana, A. (1996) in Nanofabrication and Biosystems, eds. Hoch, H. C., Jelinski, L. W. & Craighead, H. G. (Cambridge University Press), pp. 258-275, and references therein; and Jung, D. R., Cuttino, D. S., Pancrazio, J. J., Manos, P., Cluster, T., Sathanoori, R. S., Aloi, L. E., Coulombe, M. G., Czamaski, M. A., Borkholder, D. A., Kovacs, G. T. A., Bey, P., Stenger, D. A. & Hickman, J. J. (1998) J. Vac. Sci. Technol. A 16, 1183-1188). Previous electrical studies of cells have focused on external macroscopic properties, such as cell membrane responses or volume, and have primarily reflected those of large ensembles of cells (Asami, K., Gheorghiu, E. & Yonezawa, T. (1999) Biophysical Journal 76, 3345-3348; and Polevaya, Y., Ermolina, I., Schlesinger, M., Ginzburg, B. Z. & Feldman, Y. (1999) Biochim. Biophys. Acta 15, 257-71).

A number of biosensors currently in use take advantage of the electrical properties or characteristics of the biological cells in order to monitor changes therein. For example, in U.S. Pat. No. 6,051,422, a device for monitoring a cell culture compromises a cell culture chamber and a monolithic structure that includes an array of planer microelectrodes disposed on a substrate wherein each microelectrode is connected to a contact point connected to a signal generation means which produces an electrical signal. Although this device can detect electrical characteristics of a portion of individual cells in the culture it requires that a portion of the cells not adhere to the surface of the microelectrodes.

The field of micro fluidics is often viewed as the next-generation technology for rapid DNA sequencing, high-throughput drug screening, and ultra sensitive chemical analysis. However, microfluidics is largely limited by the need for an external optical detector for product analysis. Not only are many analytes of interest neither inherently fluorescent nor easily tagged with artificial fluorophores, but those analytes which are fluorescent are often subject to photobleaching and photodamage. Equally significant is the inherent difficulty in integrating an optical sensor onto a microfluidic chip.

A microscale electric impedence device for measuring electric impedence has been described by Ayliffe eta al. ((1999) IEEE J. Microelectro-mechanical Systems 8:50-57; see also International Publication No. WO 00/17630 A1 by Ayliffe et al. Published Mar. 30, 2000). The Ayliffe device has two reservoirs connected by a single microfluidic channel and ahs gold electrodes with rounded ends protruding into the narrowest portion of the microchannel (˜10 um). The device measures electric impedence over the frequency range of 100 HZ to 2 MHz.

The Ayliffe device has the limitations in that it only measures impedence, the electrodes are rounded and protrude into the micro channel, the device does measure changes in impedence over time, nor does it measure or detect single molecules or cells.

Thus, there is a need to develop microfluidic and nanofluidic and devices and chips consisting of an integrated optical sensor which can measure and record, directly and without external intervention, the unique dielectric response of material flowing through the device. Additionally, a need exists for an integrated chip can that is capable of easily identifying different fluids, including aerosols, solvents and buffers of varying ionic concentration- and particles, such as but not limited to, whole biological cells-suspended in those fluids. There is a need to provide a microfluidic device or chip capable of measuring biological properties of individual cells, such as DNA content. Further, there is a need to develop nanofluidic devices for detecting and measuring single molecules. Such devices are needed for use in quick chemical analysis, environmental and analyses and to medical diagnostics.

### SUMMARY OF THE INVENTION

The present invention relates to microfluidic and nanofluidic electrical devices for detecting or measuring an electrical property of a fluid including a liquid or aerosol, a single molecule, or a single particle or cell in a fluid. In a particular embodiment, the devices detect or measure changes in capacitance of a fluid, molecule, particle or cell as it passes through the device. The present invention also relates to the detection and measurement of single molecules, in particular, biological molecules. The present invention also relates to methods of sequencing polynucleotide molecules, such as RNA or DNA, by detecting differentially labeled single nucleotides. Applications of this technology of single molecule detection, includes DNA or RNA sequencing (requiring a resolution of 3-5 nucleotides), detection of SNPs (requiring a single nucleotide resolution), proteomics (requiring 3 nucleotide resolution), and particle sizing. The nano- and microfluidic devices of this invention also have utility for use as detectors in molecular sorting systems and detecting of pathogens, spores.

The present invention relates to microfluidic device in which characteristics of a biological cell are determined by applying an electrical signal to an individual cell and detecting signals resulting from the application of the electrical signal. The cell can be passed through a channel from a fluid input apparatus. The channel passes the cell in the vicinity of a pair of electrodes. The width of the channel, rate of flow of fluid containing the biological cell and concentration of the cells in the fluid are selected to allow cells to flow one-by-one in the vicinity of the electrodes. The microfluidic device can be used to determine the DNA content of the cell, to analyze cell-cycle kinetics of populations of the cells and as an assay for abnormal changes in DNA content of cells. The present invention is also referred to as “Capacitance cytometry”, and it has the potential to be simpler, faster, and less expensive than standard laser flow cytometry.

### BRIEF DESCRIPTION OF THE DRAWINGS

For a better understanding of the present invention, reference may be made to the accompanying drawings.

FIG. 1A is a top view of the microfluidic device in accordance with the teachings of the present invention. FIG. 1B is a side view along the vertical axis of the device shown in FIG. 1A.

FIG. 2 is a graph showing the response of the microfluidic device to different fluids; i.e. 18MΩ water, ethanol, and methanol. The graph plots the change in capacitance over time (minutes).

FIG. 3 is a graph showing the response of the microfluidic device to different ionic concentrations in the buffer 2-(N-morpholino)ethane sulfonic acid (MES) at varying pH (pH=4.52, 5.07, and 6.18). The graph plots the change in capacitance over time (minutes).

FIGS. 4A-B are graphs showing the response of the microfluidic device upon flowing cells through the device. FIG. 4A is a plot of the change in capacitance over time (minutes) and FIG. 4B is a graph of a plot in the change in conductance (ns) over time (minutes).

FIG. 5 is a graph showing the changes in capacitance (fF) over the course of 1000 ms to the passage of fluid containing mouse myeloma cells.

FIG. 6 A is a frequency histogram of the SP2/0 cells obtained with a device of h=30 μm. FIG. 6B is a comparison of a frequency histogram.

FIG. 7 is a graph of the change in capacitance C_{T} obtained by conventional laser flow cytometry vs. DNA content of mouse SP2/0, yeast, avian, and mammalian red blood cells.

FIGS. 8A-J are graphs showing DNA Progression of Rat-1, Rodent Fibroblast Cells. Figures A-E are histograms of data collected using capacitance cytometry. Standard laser flow cytometry data for the same population of cells are shown in inset Figures F-J for comparison.

FIG. 9A is a top view of a microfluidec device having a series of nano-electrodes.

FIG. 9B is side view of the microfluidic device of FIG. 9A with a PDMS coverslip.

FIG. 10 is a side view of a channel of diameter (d) and the cross sectional area (A) of the electrode.

FIG. 11A is a schematic top view of the device shown in FIG. 9A without a single molecule. FIG. 11B is a schematic top view showing a single molecule between the electrodes.

FIGS. 12A-C are a series of drawings of schematic top views of the series of electrodes for which a single molecule is passing between in the channel. FIG. 12A is at time=0; FIG. 12B is at time=t₁ and FIG. 12C is at time=t₂. FIG. 12D is a graph representing the chance in capacitance duration between time points t₁ and t₂. The length of the molecule can be calculated from the length of time the capacitance changes (increases) multiplied by the velocity the molecule is traveling through the channel.

FIG. 13 is a schematic diagram of the use of a nanofluidic device 30 shown in FIG. 9A for detecting single molecules.

FIG. 14 is a schematic diagram of of the nanofluidic channel of the nanofluidic device for use in detecting single molecules or single labeled molecules.

FIG. 15 is a graph showing the change in capacitance over time (ms) observed in response to DNA-filled lipasomes.

FIG. 16 is a photograph of an electron micrograph of microfluidic device 10.

### DETAILED DESCRIPTION

Reference will now be made in greater detail to a preferred embodiments of the invention, examples of which are illustrated in the accompanying drawings. Wherever possible, the same reference numerals will be used throughout the drawings and the description to refer to the same or like parts.

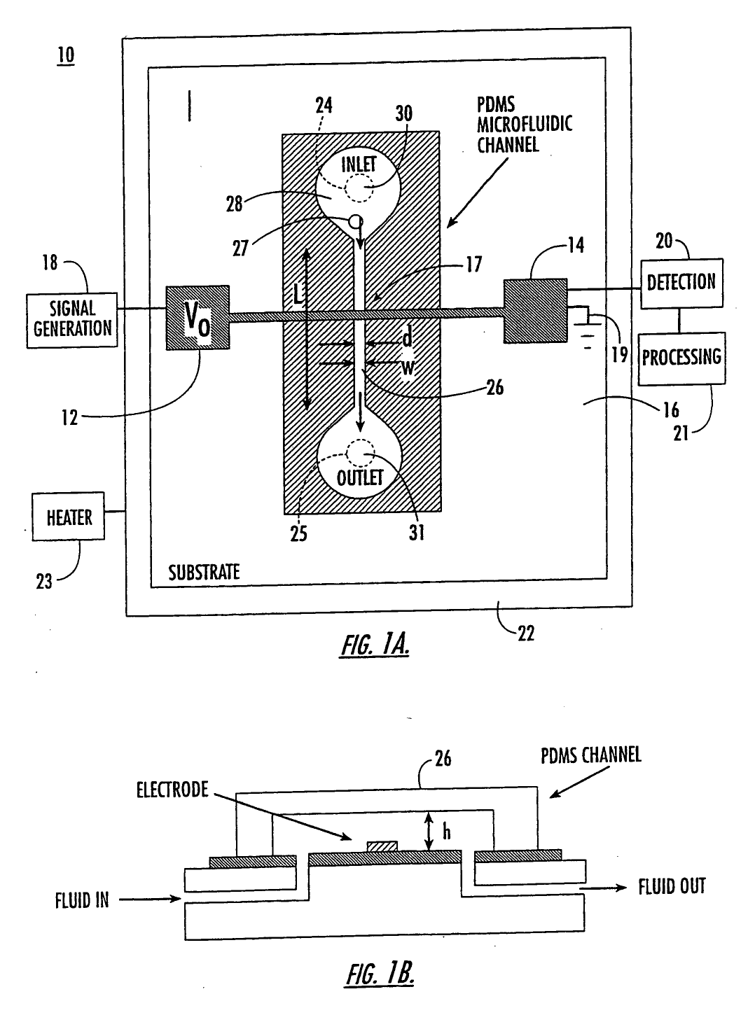

FIG. 1 is a schematic diagram of microfluidic device 10 in accordance with the teachings of the present invention. Electrode 12 and electrode 14 are disposed on substrate 16. Electrode 12 is connected electrically to signal generation means 18. Signal generation means 18 drives electrode 12 with an electric signal. The electric signal can be either a voltage signal or a current signal. Preferably signal generation means 18 generates an AC voltage. Electric field 17 is created between electrode 12 and electrode 14. The resulting signals can be detected at electrode 14 with signal detection means 20. Electrode 14 is also connected to ground 19. The detected signal is processed with monitoring and processing means 21. For example, monitoring and processing means 21 can determine various electrical characteristics such as impedance, capacitance, or conductance.

A wide range of suitable voltages can be applied to the device depending upon the sample being analyzed and its requirements for detection and measurement. Those skilled in the art are capable of determining the suitable voltages using standard electrical measurements known in the art. In one embodiment, a suitable applied voltage is between about 1 mV and 10 V. In another embodiment, a suitable applied voltage is between about 5 mV and about 1 V. In yet aanother preferred embodiment, the suitable applied voltage is between about 5 mV and 500 mV.

A wide range of applied frequencies can be applied to the microfluidic device depending upon the sample being analyzed and its requirements for detection and measurement. Those skilled in the art are capable of determining the suitable frequencies necessary using standard electrical measurements known in the art. In one embodiment, the applied frequency is between about 1 Hz and up to about 100 Ghz. In another embodiment, the applied frequency is between about 1 Hz and 50 GHz. In yet another embodiment, the applied frequency is between about 1 Hz and about 100 MHz.

In a preferred embodiment for the detection of individual biological cells, voltages of about 200 mV and about 300 mV and frequencies in the range of about 1 kHz have been found to be particularly suitable. Other voltages and frequencies can also be suitable for biological cells, and these electrical parameters can be determined empirically by those skilled in the art depending upon the cell type used and the cell's particular characteristics. The voltage can be applied at a given or substantially constant frequency.

Alternatively, a number of different frequencies can be applied over a period of time. The different frequencies applied over a period of time is useful for to pinpoint a resonant frequency. Those skilled in the art of microfluidic electrical devices are capable of determining the range of frequencies applied and the length of time it is necessary to apply such frequencies. In one embodiment, the frequency range applied ranges from about 1 Hz to about 100 Ghz. In other embodiments, the frequencies range from about 1 Hz to about 50 Ghz, about 1 Hz to about 1 GHz, and from about 1 Hz to about 750 Mhz. In yet another embodiment, the frequencies range from about 1 Hz to about 500 MHz. The length of time the range of frequencies is applied depends upon the size of the range of frequencies, i.e. the large the range of frequencies the longer the time period is required for the “sweep.” For example, but not by of limitation, a frequency sweep from 1 Hz to 500 MHz can require greater than five minutes.

The capacitance or conductance is preferably measured using an AC bridge. A number of suitable AC bridges are currently manufactured and commercially available. For example, but not by way of limitation, a suitable AC bridge is manufactured as described in Andeen Hagerling as a AH2500A 1 kHz Ultra-Precision Capacitance Bridge. In one embodiment, this bridge applies a voltage (Vₙₘₓ=250 mV) at a frequency of 1 kHz across electrode 12 and electrode 14. In this embodiment, a Vₙₘₓ=250 mV is applied such that it represents a small pertubation to the entire system. At this voltage, electrolysis and dielectrophoretic trapping can be ignored as both occur at higher voltages Vₘₓ>1 V. Use of conventional techniques for electrically shielding. the device and controlling the temperature precisely make it possible to achieve suitable noise levels. The in-phase and quadrature response of electrodes 12 and 14 are compared to those of a reference capacitor within the bridge to determine the total capacitance C_{T} and loss R in the system.

Capacitance measurements performed using an Andeen Haegerling capacitance bridge, while extremely sensitive, this bridge measures only at 1 kHz frequency. While this frequence is the preferred frequency for measuring DNA content of cells, other frequencies can be optimal for measuring other cellular components or individual molecules. Other preferred methods of measuring capacitance are to adapt the microchip design to interface with both a network analyzer and an impedence analyzer. These analyzers are used with other side ranges of frequency (kHz to GHz) to detect and measure multi-component analytes, i.e., it will become possible to perform dielectric spectroscopy. Such a device has been made.

Alternatively, other types of instrumentation for measurement of AC conductance or capacitance can be used. Other AC bridges and different sizes of electrodes and microfluidic channels can be chosen to apply higher voltages to the microfluidic devices of the present invention.

In alternative embodiments of the invention, the device comprises data acquisition means 50. Since a commercial capacitance bridge was used in some preferred embodiment, the data was acquired every ˜100 milliseconds. In other embodiments of the invention, such data acquisition means are “lock-in”s such as a PAR 124 lock-in amplifier with the reference capacitor fabricated on the microchip. Such an arrangement allows data acquisition every 20 milliseconds, and the detection throughput is increased from 15-20 cells/sec to ˜100 cells/sec. Alternatively, other data acquisition means known in the art can be used with compatible capatiance analyzers.

Temperature of microfluidic device 10 is controlled using methods commonly used in the art, such as by mounting substrate 16 on temperature conductive block 22. For example, temperature conductive block 22 can be formed of a mineral such as, but not limited to, quartz, glass, Al₂O₃, polyamide and sapphire. Temperature conductive block 22 is connected to heater 23 for controlling the temperature to within suitable range limits, for example, within about 0.05° C. In one embodiment of the invention, noise levels of about ˜5 aF when the microfluidic channel is dry and about 0.1-2 fF when wet were achieved using a heater 23.

During operation, as electrode 12 is driven with the applied voltage signal current flows between electrode 12 and electrode 14. Inlet 24 receives a fluid 28 from fluid input means 29. Inlet 24 and outlet 25 are in communication with channel 26. Fluid 28 flows from inlet 24 through channel 26 to outlet 25.

The fluid input means encompasses any means known or to be known that enable the fluid to move through the microfluidic channel 26. For example, but not by way of limitation, the input fluid means 29 are devices that force fluid 28 through the inlet 24 and outlet 25 using pressure. Alternatively, the input fluid means 29 are devices that force fluid through the inlet 24 and outlet 25 using electric fields. In one embodiment, the imput fluid means 29 is a syringe pump (such as but not limited to, the KD Scientific Syringe Pump, Model KD2100) to deliver fluid 28 through the device at non-pulsating rate ranging from about 1 μl/hr to about 300 μl/hr. In a preferred embodiment of the invention, the fluid 28 is delivered in a non-pulsating manner in order to avoid fluctuations in electrical (i.e., capacitance) measurements due to fluctuations in the presence of fluid in the channel 26.

The word fluid 28 is defined as any liquid or aerosol. The fluid 28, is exemplified, but not limited to, liquids, such as water, organic solvents, cell cultures, animal or human bodily fluids, solutions comprising particles, solutions comprising biological molecules, cellular cytoplasm, cellular extracts, cellular suspensions, solutions of labeled particles or biological molecules, solutions comprising liposomes, encapsulated material, or micelles, etc. As understood in the art, a liquid “is the state of matter in which a substance exhibits a characteristic readiness to flow, little or no tendency to disperse, and relatively high incompressibility” (The American Heritage Dictionary, New College ed., (1982) p. 761). A fluid is also understood to encompass aerosols, which are defined as “a gaseous suspension of fine solid or liquid particles” (Id., p. 20). For example, aerosols are, but not limited to, gaseous suspensions of liquids, particles, cellular components or molecules, cells, and any molecules capable of comprising an aerosol state.

In another embodiment of the invention, fluid 28 further comprises particles. As used herein, particles are defined as any small amount of material capable of causing a change in electrical characteristic of the fluid (i.e. capacitance or conductance) when the fluid comprising particles flows through microfluidic or nanofluidic devices 10 and 40, respectively. By way of example, but not by way of limitation, particles are any polymer particle, such as polystyrene particles or beads, metal colloids (e.g., gold colloidal particles), magnetic particles, dielectric particles, nanocrystals of materials, and bioparticles, such as spores, pollen, cellular oclusions, precipitates, intracellular crystals, etc. In embodiments of the invention, the particles are in the nanometer and/or micrometer size range, for example, but not limited to, from about 1 nm to about 100 μm.

In still another embodiment of the invention, the fluid contains biological molecules, such as but not limited to, polynucleotides such as DNA and RNA, polysaccharides, polypeptides, proteins, lipids, peptidoglycan, and any other cellular components. The microfluidics and nanofluidics devices of the invention are capable of detecting biological molecules in a fluid sample.

In an embodiment of the invention, the fluid 28 comprises viruses, such as but not limited to, viruses capable of infecting any organisms including microorganisms, plants, or animals, in particular, mammals, and preferably humans. Any virus capable of being detected by an alteration in the electrical characteristics of a fluid 28 is encompassed by the present invention. Further, viruses in the categories of viruses with or without coat, and viruses categorized as DNA or RNA viruses, either double stranded or single stranded are encompassed by the present invention.

In yet another embodiment, fluid 28 further comprises one or more biological cells 27. The biological cells are either procaryotic and/or eucaryotic cells. Examples of procaryotic cells include, but are not limited to, bacteria, microorganisms, etc. Preferably, biological cells are eucaryotic cells having a nucleus. Examples of eucaryotic cells are, but not limited to, fungal, plant, and animal cells. In particular, the cells are mammalian cells, most preferably human cells. In more particular embodiments, the mammalian or human cells may be cells for example, from blood, liver, kidney, lung, or any other tissue or organ. In another preferred embodiment, the cells are tumor or cancer cells, which can be either benign or malignant cancer cells.

The rate of flow of the fluid 28 by the fluid input means 29 and size of the channel 26 are selected to allow sufficient time for the device to detect and/or measure the electrical characteristic(s) of the fluid or component of interest (i.e., particle, molecule, cell, etc.). Also, the concentration of the fluid or the concentration of the components of interest in the fluid 28 (such as the biological cells in biological cell bearing fluid), is selected to allow the components in the fluid 28 to flow one by one through channel 26 and be individually monitored by monitoring and processing means 21.

In a preferred embodiment, one or more of the factors of concentration of biological cells in biological cell bearing fluid 28, rate of injection of biological cell bearing fluid 28 by fluid input means 29 and size of channel 26 are selected to allow biological cells 27 suspended in biological cell bearing fluid 28 to flow one by one through channel 26 and be individually monitored by monitoring and processing means 21.

Inlet 24 and outlet 25 can be formed of respective apertures 30, 31 drilled through substrate 16. A wide range of suitable sizes of inlet 24 and outlet 25 are possible, and those skilled in the art are capable of empirically determining the preferred size ranges depending upon the fluid 28 or fluid component to be analyzed. The size of the inlet or outlet is not a limiting factor. By way of example, a suitable size of inlet 24 and outlet 25 is an aperture having a diameter of about 1 mm to about 3 mm for evaluating whole biological cells. Channel 26 is selected to have a width and height which are substantially the same or a predetermined amount larger than a diameter of the particle or biological cell 27 to allow them to flow one by one through channel 26. Distance d separating electrode 12 and electrode 14 is selected to correspond to width w of channel 26 in order to avoid complications arising from a particle or biological cell passing directly over only one electrode and to minimize the effects of electrode polarization.

By way of example, and not limitation, a suitable distance d can be in the range of 10 nm to 1 mm. Channel 26 can have a width w and height h in the range of 1 nm to 1 mm. Preferably, distance d separating electrodes and width w of channel 26 are in the range of about 15 nm to about 50 μm. Height h of channel 26 is preferably in the range of 15 nm to 60 μm. For example, for about a 10 μm average diameter eukaryotic cell a suitable distance d for about a 50 μm wide electrode channel and width w is about 30 μm and channel height h is in the range of about 30 μm to about 40 μm.

Substrate 16 can comprise a variety of materials, such as silicon, glass, metal, quartz, plastic, ceramic, polyethylene or any suitable type of polymer. For example, electrodes 12 and 14 and their respective interconnectors to signal generation means 18 and signal detection means 20 can comprise any biocompatible conductive substance such as gold, titanium, copper, platinum, iridium, polysilicon, carbon or aluminum.

Electrodes 12 and 14 are fabricated, for example, with photolithography or electron-beam lithography. Known photolithographical etching techniques can be used to form channel 26 on substrate 16. Alternatively, channel 26 can be formed as a molded part with soft lithography techniques as described in Xia, Y., Kim, E., and Whitesides, G. M. ((1996), Micromolding of Polymers in Capillaries, Applications in Microfabrication. Chem, Mater. 8:1558-1567), hereby incorporated by reference into this application. Photolithography can be employed to define a “master” of channel 26. Replicas of the master are created using an elastomeric polymer material, such as polydimethylsiloxane (PDMS). The surface of PDMS can be oxidized using a de-generated oxygen plasma to provide a charged, hydrophilic silicon-oxide surface. Channel 26 is aligned to and positioned on top of or between electrodes 14 and 16. Direct contact between channel 26 formed of PDMS and substrate 16 provides a hermetic seal.

In another embodiment, the microfluidic device 10 is fabricated by embedding the electrodes in the substrate.

In yet another embodiment, the channel 26 is etched and the electrodes are made on sides of walls to create a parallel-plate capacitor device.

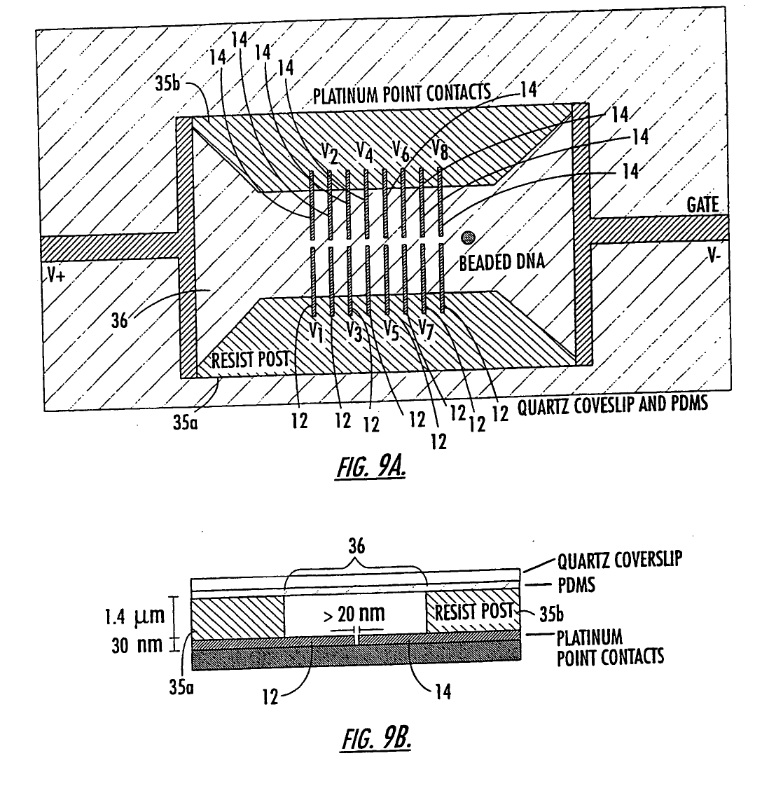

In another embodiment of the invention, the microfluidic device 10 comprises a plurality of electrode pairs 12 and 14 arranged to form channel 26 between the pairs. As shown in FIGS. 9A-B, 11 and 12A-C, the electrodes 12 and 14 are arranged in parallel along the length (L) of channel 26. For example L can be in the range of about 1 nm to about 10 mm. The series of electrode pairs along the length are useful for measuring the rate at which particles flow through the channel 26 and, in particular, are useful for determining the length or size of molecules. In an embodiment of the invention post 35A is formed on electrode 12 which permits the introduction of fluids 27 to the channel 26 and enables the delivery of single molecules. Post 35B is formed on electrode 14. Channel 36 is formed between post 35A and post 35B. Channel 36 has a width W larger than the distance d between electrodes 12 and 14. For example, width W can be in the range of about 1 μm to about 100 μm. An example of determining the length of a molecule is set forth in Example 6.

Those skilled in the art of microfluidic and nanofluidic electrical fabrication will be able to use a variety of methods to manufacture the devices of the present invention.

Basically, the devices of the present invention function as follows: fluid 28, particle X, or biological cell 27 (collectively, also referred to as the “fluid sample”) passes through electric field 17. Impedance between electrode 12 and electrode 14 is determined from the current signal. Based on the impedance, various electrical characteristics of the fluid sample passing through electric field 17 are determined by monitoring and processing means 21. Such electrical characteristics include impedance of the fluid sample and change of capacitance ΔC_{T} before and after fluid sample enters electric field 17, and conductance. Roughly, electrodes 12 and 14 act as a parallel-plate capacitor with a capacitance C given approximately by

$$C = \frac{A {ɛ}_{o} κ}{d}$$

where A is the cross-sectional area, d is the separation width, ε₀ is the permittivity of space (8.85*10⁻¹² Farads), and K is the dielectric constant of any material between electrodes 12 and 14. More specifically, the present configuration of the device is measuring the stray capacitance which is in the range of about pF and greater. In air, the capacitance of electrodes 12 and 14 is approximately Cₐᵢᵣ≅0.27*10⁻¹⁸ Farads (F). When water flows through channel 26 and fills the gap between electrodes 12 and 14, the capacitance increases by almost two orders of magnitude to C_{water}≅21*10⁻¹⁸ F. A change of capacitance ΔC_{T} defined and scales with the dielectric constant of the fluid C_{T} (final)-C_{T} (initial) which can be used to measure the change is capacitance as fluid sample (fluid 27) pass through electric field 17. The electrical characteristics of biological cells can be determined by modeling the biological cell with conventional techniques. For modeling particles or cells, various models can be used such as modeling the particle or cell as a flat circular pancake, square, rectangle, sphere or cube.

### Nanofluidic Devices

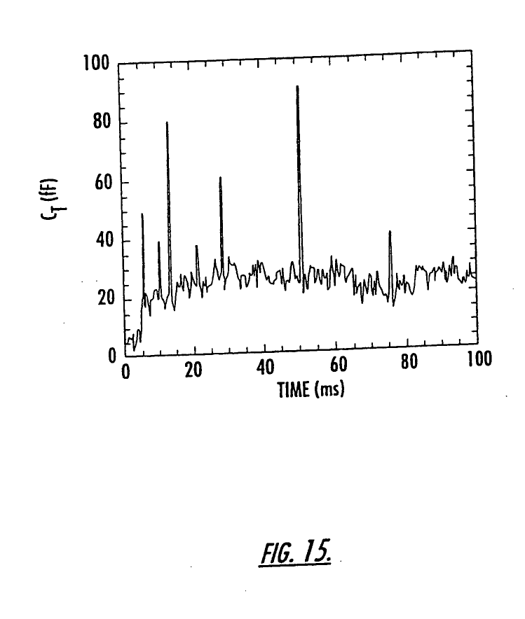

The present invention also provides for nanofluidic devices. Nanofluidic devices 40 are of the same design as the microfluidic devices 10 described above, except that they are characterized by having dimensions of electrodes 12,14 and channel 26 in the nanometer ranges, such as between about 10 nm to about 100 nm. By way of example, the nanofluidic devices comprises one or more pairs of electrodes 12 and 14 having a length and width of the electrode in the nanometer ranges. For example, the electrode ends are about 30 nm wide and about 20 nm high. Further, nanofluidic device 40 comprises an electrically insulating layer 42 grown on the substrate to electrically isolate the electrodes. The electrically insulating layer 42 can be any electrically insulating material used in microelectromechanical devices, such as, but not limited to, Si₃N₄. Electrode insulating later 42 is supported by temperature conductive block 22 connected to heater 23. Shielded box 44 surrounds device 40 and temperature conductive block 22 for providing electrical shielding. Capacitance bridge 45 is coupled to device 40 for providing capacitance measurements. Nanofluidic device 40 is further characterized in that the electrode pairs 12 and 14 lie within a larger channel 36 described above. For example, channel 36 can be in the range of about 5 to about 10 μm. As shown in FIG. 14, individual molecules flow through the smaller channel 26 between the electrodes 12 and 14, or pairs of electrodes, for detecting molecules and/or measuring their size.

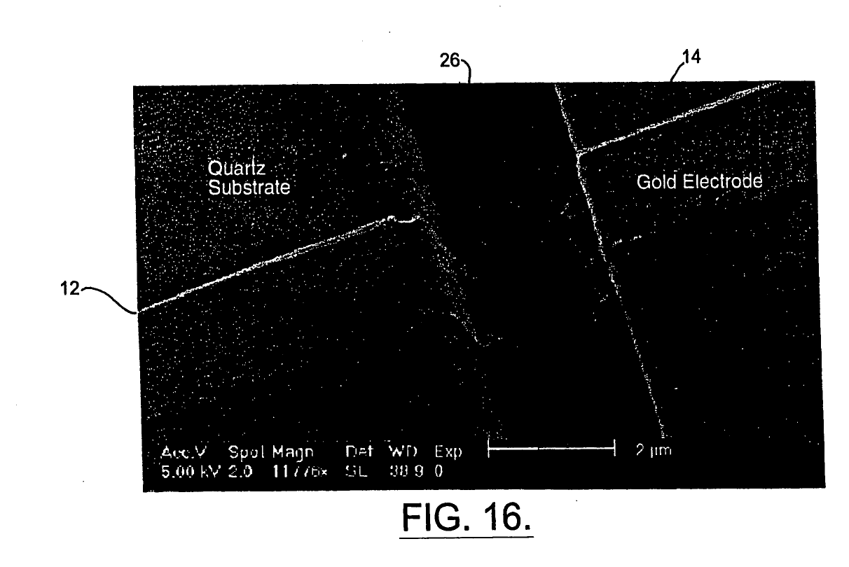

FIG. 16 is an electron micrograph. Electrodes 12 and 14 are substantially flat having a rectangular shape. Electrodes 12 and 14 are formed of a conductive material such as gold. Channel 26 is etched in substrate 16. For example channel 26 can have a width of about 2 μm. Residual conductive material can be removed using conventional reactive-ion etch.

The nanofluidic devices of the present invention are particularly useful for detecting and measuring changes in electrical properties of fluids and single molecules. In a preferred embodiment, these nanofluidic devices are used for determining the nucleotide sequence of polynucleotide molecules using labeled nucleotides as described below.

### Methods of Use

In another embodiment of the invention is a method of determining the capacitance of a fluid or detecting a fluid in the device. In one embodiment, the method comprises the steps of passing a fluid 27 through the channel of the microfluidic or nanofluidic device described above. The fluid passes through the electrical filed 17 and impedence between one or more pairs or electrodes 12 and 14 is determined from the current signal. The electrical characteristic of the fluid is determined by monitoring and processing means 21. The change in the electrical characteristics of the fluid is determined by the devices described above. An example of the use of the device and method is set forth below in Example 1.

Another embodiment of the invention provides for methods of detecting differences in ionic concentration of fluids 27. In this embodiment, the fluid 27 passes through the channel 26 between the electrodes 12 and 14. If there is a difference in the electrical characteristics of the fluid or fluids, the difference can be detected or measured as a difference in capacitance. The difference in capacitance can be converted to differences in ionic concentration by comparing the test fluids with a standard curve generated using fluids of known ionic concentration. An example of this method is set forth below in Example 2.

The invention is also directed to methods of detecting biological molecules, and determining the size or length of such molecules. In a preferred embodiment, the microfluidic or nanofluidic devices are used to detect single molecules due to changes in capacitance as they flow between one or more pairs of electrodes.

As shown in FIG. 9, a preferred device consists of a series of pairs of metallic electrodes nanofabricated onto a silicon or III-V based substrate. Other substrate materials may also be used without altering functionality. Each electrode in FIG. 1 is 30 nm wide and 20 nm high, with a 10 nm gap between the constituents of each pair. We are not limited to 10 nm gap separations between electrodes. In fact, we can have gap separations ranging from 1 nm to several microns wide. An electrically insulating layer such as Si₃N₄ is typically grown on the substrate to electrically isolate the electrodes. The electrodes lie within a larger channel which permits the introduction of fluid to the capacitors and enables the delivery of single molecules.

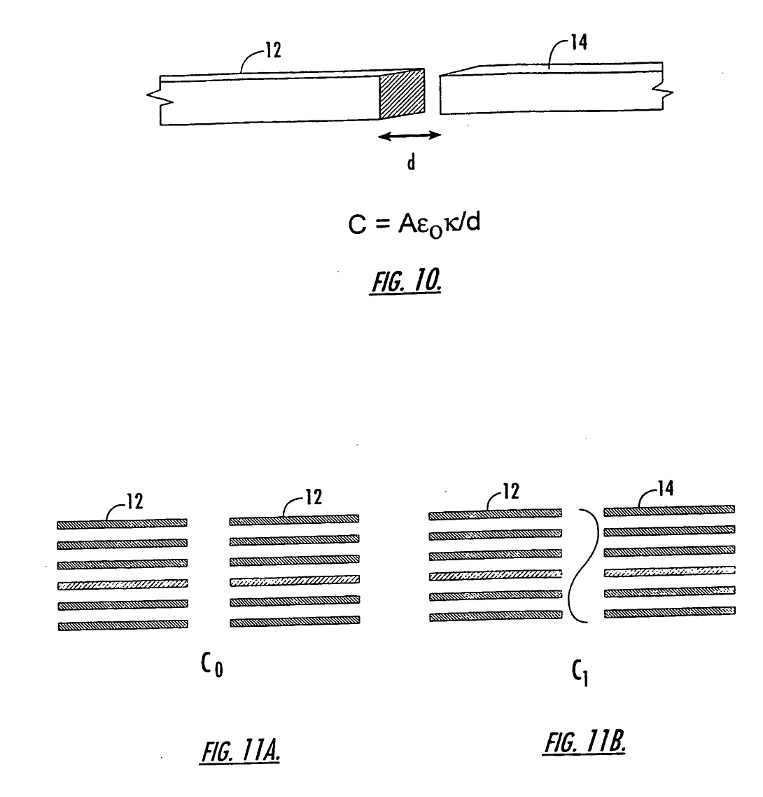

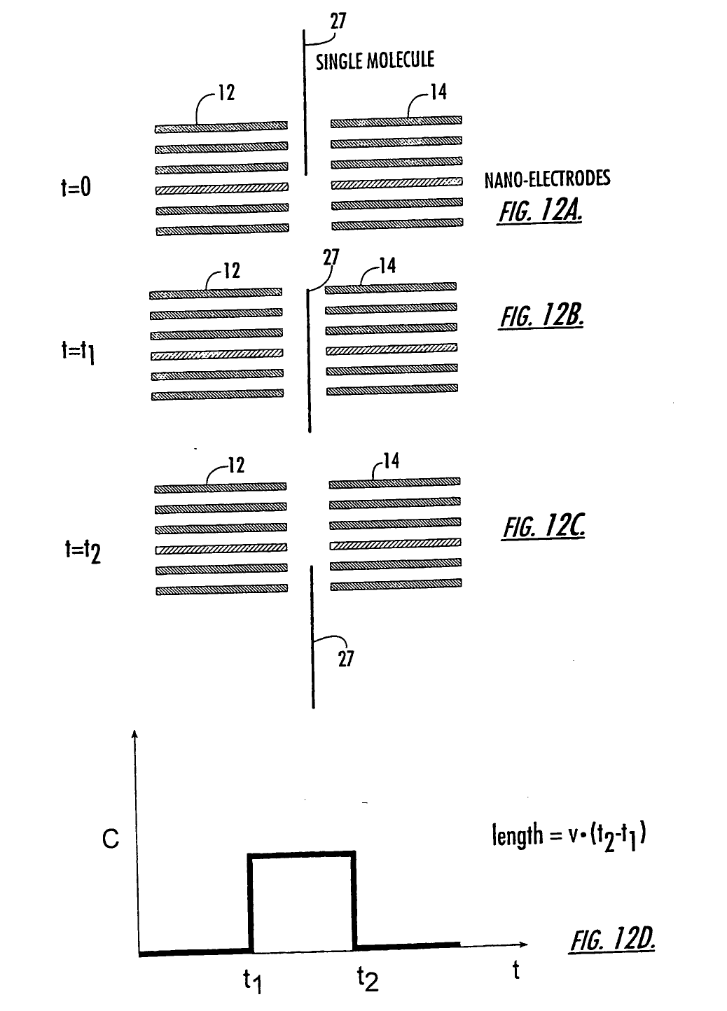

Each pair of electrodes acts as a parallel-plate capacitor with a capacitance C given approximately by

$$C = \frac{A {ɛ}_{o} κ}{d}$$

Where A is the cross-sectional area, d is the separation width ε₀ is the permittivity of space (8.85*10⁻¹² Farads), and κ is the dielectric constant of any material between the plates (see FIG. 10). In air, the capacitance of each pair of electrodes in FIG. 1 is Cₐᵢᵣ21*10^{-18F}. Almost any liquid with known K can be used. For example, solvents such as toluene and alcohol are used because single molecules of DNA are insolvent in such liquids. Capacitance can be measured with a variety of techniques, with the most sensitive yielding precision exceeding 0.1*10⁻¹⁸ F, which is amply sufficient to detect changes of this size.

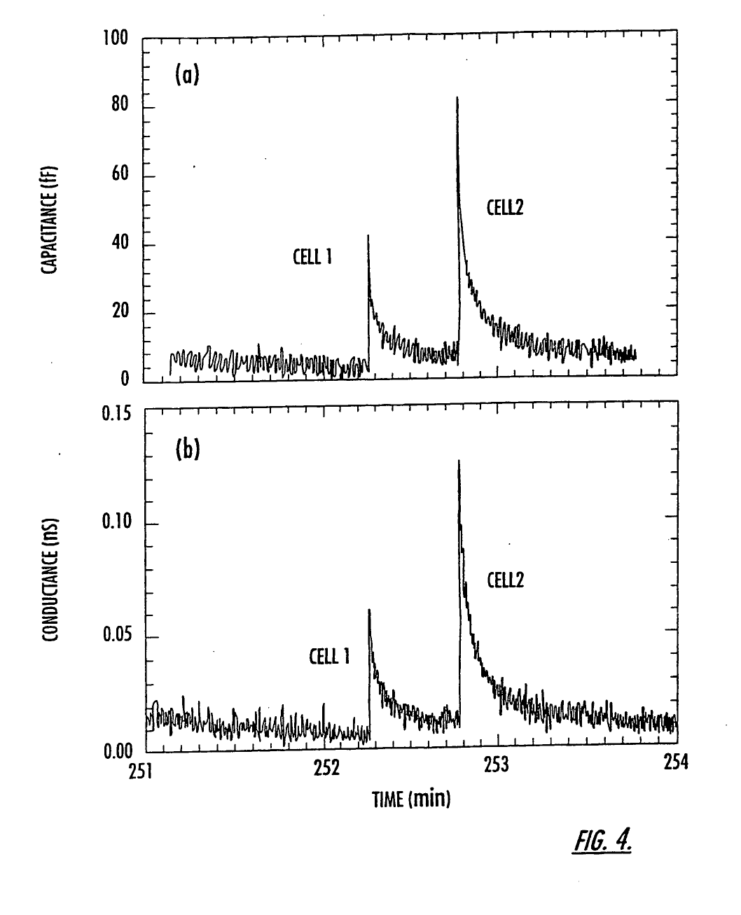

Single molecules can vary the capacitance between nano-electrodes in much the same way as water or any other dielectric material. By hydrodynamically focusing and stretching single molecules into the array of nano-electrodes, we can measure the capacitance and compare it to that when only liquid flows through the device (see FIG. 4). The change in capacitance is due to the presence of single molecules;

thus, there is a means of detecting non-optically single molecules.

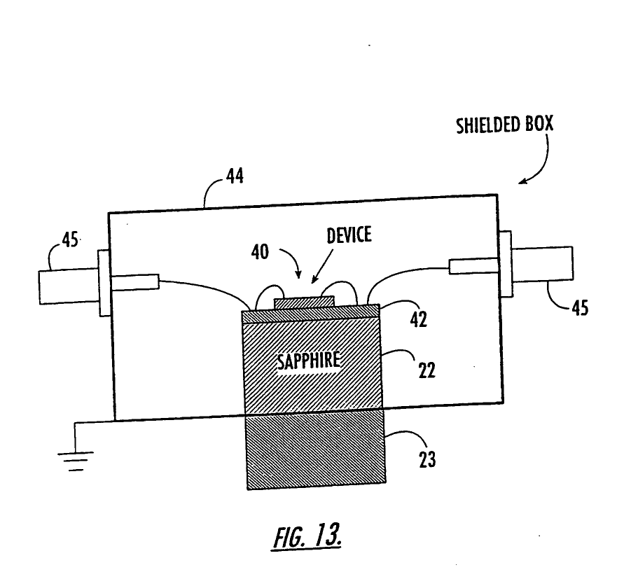

In another embodiment, the length of molecules is determined by performing passage of time measurements and knowing the velocity at which the molecules flow, we can also determine the length of the single molecules (see FIGS. 12A-D). FIGS. 12 A-C are schematic drawings of a molecule passing through a nanofluidic channel 26 between a series of pairs of electrodes. FIG. 12D is an example of the change in capacitance measurements expected as the molecule flows through the pairs of electrodes. Determining the length of molecules becomes increasingly important when performing single molecule manipulation, sequencing, hybridization, or local molecular reactions. An example of detecting and measuring molecules is set forth in Example 6.

The results are two-fold. First, with this device, we have the means of non-optical detection and length determination of single molecules. Second, the pairs of nano-electrodes can be fabricated into many arrays on a single 300 mm wafer, thus allowing for parallel measurements and sorting.

The present invention also provides for an integrated “chip” having microfluidic devices for detecting and measuring molecules. In another embodiment, the chip comprises a plurality of microfluidic devices capable of determining changes in capacitance for detecting and measuring molecules. In another embodiment, the integrated chip comprises a plurality of microfluidic devices capable of detecting and measuring molecules, and devices for sorting such identified molecules, wherein the integrated chip identifies and sorts molecules of interest. For example, the integrated chip is capable of sorting a mixture of polynucleotides or protein, or any other molecules of interest.

In a preferred embodiment, microfluidic device 10 is used to detect the presence of biological cells 27 in a fluid 28. By way of example, a suitable range of concentration of biological cells 27 in biological cell bearing fluid 28 is about 10³ to about 10¹⁰ cells/ml. However, the concentration of biological cells in the fluid and flow rate can be adjusted so as to have individual single cells pass between the electrodes.

Fluid input means 29 delivers fluid comprising biological cells 28 to inlet 24 at a rate, for example, but not by way of limitation, in the range from about 1 μl/hr to about 300 ml/hr. Those skilled in the art are able to adjust the flow rate accordingly to be even greater flow rates. For example, fluid input means 29 can be a syringe pump such as manufactured by KD Scientific Syringe Pump as Model KD2100. The fluid input means also encompasses pressurized devices or electrical field devices to create flow of the fluid. Preferably, the concentration of biological cells in biological cell bearing fluid 28 is in the range of about 10⁵ to about 10⁶ cells/mL, and fluid input means delivers biological cell bearing fluid 28 at a rate of 1 μL/hr to about 5 mL/hr to provide a dilute concentration flowing at a slow rate to allow biological cells 27 in biological cell bearing fluid 28 to flow one by one through channel 26. An example of using the microfluidic device 10 to detect cells in a fluid is set forth below in Example 4.

In a preferred embodiment of the invention, microfluidic device 10 is used in a method of quantifying the DNA content of single eukaryotic cells. DNA content of individual eukaryotic cells can be determined with microfluidic device 10. The greater the DNA content of the cell the greater the impedance or capacitance. The position of such a cell along the mitotic cell cycle is strictly related to DNA content such that a cell in G₀/G₁-phase has 2N DNA content, a cell in G₂/M-phase has 4N DNA content, and a cell in S-phase has between 2N and 4N DNA content. Because DNA is a highly charged molecule, the phase of an individual cell can be determined from a change in capacitance which approximately scales with the DNA content of the cell.

Accordingly, the system 10 response ΔCᵣ to a cell in G₂/M-phase should be roughly twice that in G₀/G₁-phase, as the former has twice the DNA content (4N vs. 2N DNA content); the response to a cell in S-phase should be between that of the G₀/G₁-G₂/M-phases; and finally, the response to a hyperdiploid cell (greater than 4N DNA content) should be greater than that of either a G₀/G₁-, S-, or G₂/M-phase cell. Monitoring and processing means 21 can be used for monitoring the DNA content of populations of cells to produce a profile of their cell-cycle kinetics. Examples of measuring the DNA content of individual cells are set forth below in Examples 4 and 5.

In yet another embodiment of the invention, microfluidic device 10 is used in a method of determining the cell-cycle kinetics of a population of cells. Once the DNA content of the individual cells in a population has been determined, an analysis of the population can determine the percentage of cells in each particular stage of the cell cycle. An example of this method of using the microfluidic device is set forth below in Example 5.

Another embodiment of the invention is a method of determining abnormal changes in DNA content or cell-cycle kinetics in a population of cells from a patient. Abnormalities in DNA content or cell-cycle kinetics are frequently encountered in neoplastic or cancer cells. Abnormal is defined as a measurable difference in the DNA content, percentage of cells in the G₀/G₁-, S-, or G₂/M phases of the cell cycle, or in the persentage of cells in the sub-G, population of cells, all in comparison with measurements made in a population of reference cells of the same lineage or with measurement values recorded in the medical or scientific literature.

Examples of such neoplastic or malignant cancer cells are: leukemia cells (many varieties) from the blood stream or bone marrow; shed cells from solid tumors, such as head and neck tumors, lung, colon, or bladder cancer; cells from any solid tumor, obtained by biopsy or at surgery.

Another embodiment is a method of detecting a malignant cell in a population of cells comprising the steps of determining the cell-cycle kinetics in a population of normal cells of a particular type, and the cell-cycle kinetics of a population of test cells, and comparing the cell-cycle kinetics. The abnormal or malignant cells are characterized in that the DNA content of a single neoplastic cell may be greater (hyperdiploid), lower (hypodiploid) or the same as a normal cell of the same lineage in the same phase of the cell cycle. In addition, populations of neoplastic cells may be judged abnormal by reference to the relative percentages of cells in the various phases of the cell cycle.

Another method of detecting a malignant cell in a sample of cells comprises the steps of determining the DNA content of individual cells in a sample using the microfluidic device of the present invention, and comparing the DNA content of non-malignant cells to the sample cells.

The present invention also discloses microfluidic devices and methods of DNA and RNA sequencing of single polynucleotide molecules as well as the detection and identification of nucleic acids and proteins at different levels of resolution. As described above, the fundamental basis of this technology is a capacitance measurement. As schematically shown in FIG. 14, polynucleotide molecules comprise differentially labeled nucleotides wherein the labels comprise semiconductor or differently-sized gold nanoparticles. Each nanoparticle corresponds to a different capacitance/conductance response which is measured using the pair or pairs of nanoelectrodes.

The microfluidic device for detecting single polynucleotide molecules is similar to the microfluidic devices described above. However, for detecting single molecules, the distance d separating the two electrodes is approximately 100 nm. Hydrodynamic focusing will stretch out single molecules of DNA and direct them in between the electrodes. (Knight, J. B., et al., (1998) “Hydrodynamic focusing on a silicon chip: Mixing nanoliters in microseconds” Phys. Rev. Left. 80:3863-3866) Fluid delivery within a microfluidic channel is accomplished through pressure or electric fields.

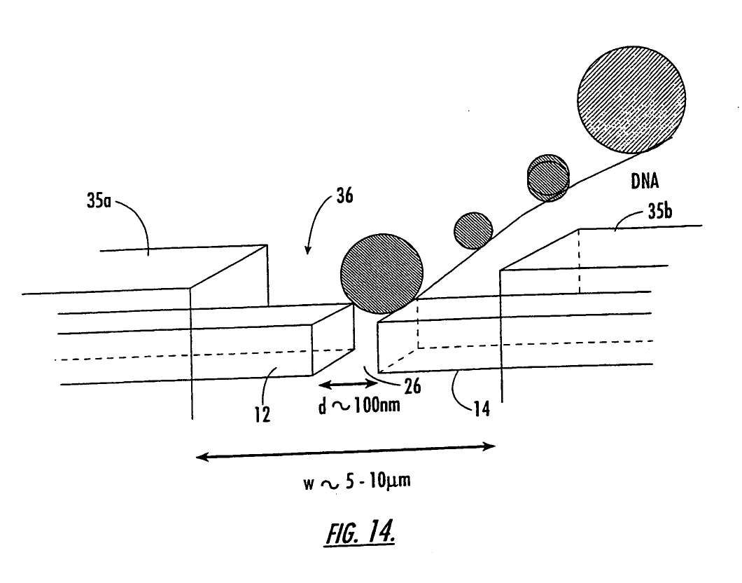

The change in capacitance as the molecule passes between the electrodes is measured using a capacitance bridge and/or lock-in amplifier in combination with a frequency source that is capable of sweeping through a range of frequencies 9 kHz to 10's GHz) during measurement. Electric shielding and temperature control are necessary for precise measurements (as shown in FIG. 13).

With increased sensitivity (via smaller electrodes, narrower microfluidic channels, and resonant frequencies) capacitance measurements are used to detect and discriminate between the differentially labeled DNA bases. The method of DNA sequencing is performed by first labeling the polynucleotide (DNA or RNA molecule) with small beads of specific dielectric properties, such as functionalized semiconductor or gold nanocrystals. Alivisatos et al. ((1999) Materials Research Society Meeting, Boston, Mass.) recently demonstrated that different semiconductor nanocrystals can be attached to specific nucleotides along a short oligonucleotide. In the present invention, to detect the four possible nucleotides (Adenine, Cytosine, Quanine, Thyamine) four different semiconductor nanocrystals or differently-sized gold particles which have different dielectric properties are used. The nucleotide sequence is “read” by detecting and measuring the different nanocrystals or gold particles using the microfluidic device. The device and method permit a resolution of a single nucleotide base for sequencing polynucleotide molecules. As shown in FIG. 14, different size particles are attached to polynucleotide molecules for resolving the nucleotide sequence. Each nanoparticle corresponds to a different capacitance/conductance response which is measured using a set of metallic electrodes.

The following examples are presented for purposes of illustration only and are not intended to limit the scope of the invention in any way.

### EXAMPLE 1

### Detecting Capacitance of Fluids

Microfluidic device 10 of the present invention is capable of detecting the capacitance of different fluids. The microfluidic device used for this experiment is shown in FIG. 1 having d 30 μm and h=30 μm.

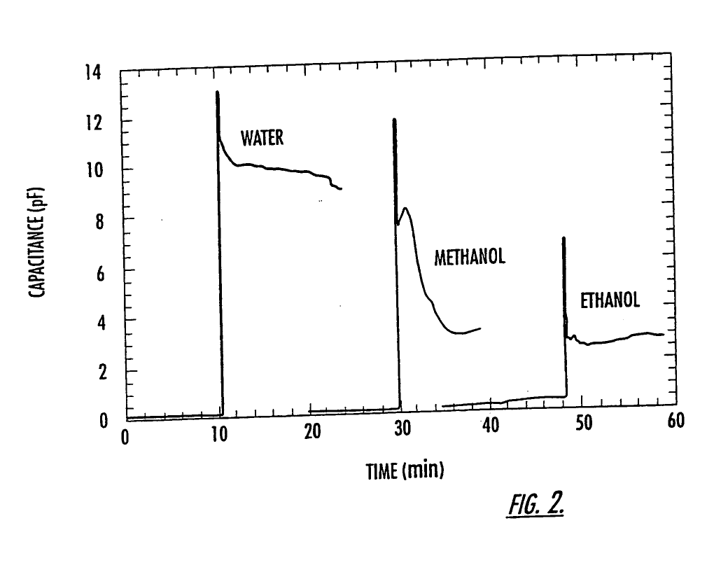

As shown in FIG. 2, there is a dramatic increase in both C_{T}, and R once fluid flows past the electrodes (C_{Tinitials} measures 0.10 pF when the electrodes are dry). Although some settling time is needed for the device, the final values of C_{Tinitials} are 9, 3 and 2.5 pF for water, methanol, and ethanol, respectively. As expected, the change in capacitance ΔC_{T}, scales with the dielectric constant of the fluid, i.e. (C_{T(final)}−C_{T(initial)})/C_{T(initial)}=for water is ˜90, that of methanol ˜30, and that of ethanol ˜25. Deviations from the actual dielectric constant of the three different solvents (water=80, methanol=33, and ethanol=24) are a result of stray capacitances, some of which are not affected by the fluid. These results demonstrate that the microfluidic device is capable of detecting differences in capacitance between different fluids and measuring the fluid dielectric constants.

### EXAMPLE 2

### Detecting Different Ionic Concentrations

Microfliudic device 10 of the present invention was used to detect differences in ionic strength of fluids. The microfluidic device used for this experiment is that shown in FIG. 1.

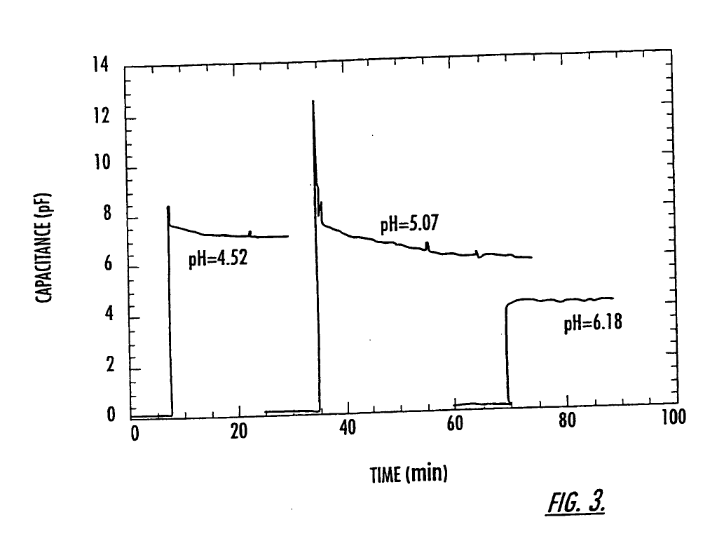

FIG. 3 shows the sensitivity of our device to different ionic concentrations. The buffer 2-(N morpholino) ethane-sulfonic acid (MES) was used at varying pH (pH −4.52, 5.07, and 6.18). As shown in the figure, the device is able to distinguish the different fluids and measure differences in ionic concentrations.

### EXAMPLE 3

### Detection of Single Mouse Myeloma Cells in a Fluid and Correlation Between Capacitance and Cellular DNA Content

The microfluidic device shown in FIG. 1 was used to detect individual, single cells and determine the content or amount of DNA in the individual cells. Also, the cell cycle stage for individual cells within a population of cells was determined and compared using flow cytometry and capacitance cytometry of the present invention. The cell cycle stage of the individual cells is determined by measuring the cellular DNA content.

Mouse myeloma cells (SP2/0), a malignant cell line, were grown in suspension to a density of approximately 10⁵ cells/mL. The cells were then washed in phosphate-buffered saline (PBS) solution (pH 7.4), fixed in 75% ethanol at −20° C. for a minimum of 24 hours, washed again with PBS solution, treated with RNAase, and then washed and resuspended for storage in 75% ethanol. Standard analysis (FACScan flow cytometer, Becton Dickinson Immunocytometry Systems, San Jose, Calif.), following treatment with a nucleic acid probe (SYTOX® Green Nucleic Acid Stain, Molecular Probes, Eugene Oreg.), showed that approximately 41% of the cells were in G₀/G₁-phase, 40% in S-phase, 18% in G₂/M phase of the cell cycle, and <1% were hyperdiploid, as determined by DNA content.

For any given experimental run, microliters of fixed cells at a concentration of 10⁵ cells/mL were injected into the microfluidic capacitance cytometer device of the present invention at a rate of 1 μL/hr. Using cells tagged with a fluorescent probe (SYTOX® Green Nucleic Acid Stain) it was visually confirmed that, at this dilute concentration, cells flowed one-by-one through the microfluidic channel at an average cell velocity of ˜250 μm/sec. (For a movie of the cells flowing through the microfluidic channel, see http://oberon.princeton.edu). The Reynolds number (Re) was estimated to be Re ˜10⁻², thus ensuring that flow in the channel was laminar.

FIG. 4A-D shows the responses we observed when running the cells through the microfliudic device. As shown in the FIG. 4A, there are two distinct peaks-corresponding to two different cells- whose final capacitance values are C_{T}-C_{T˜.40} fF and C_{T}˜80 fF. These two capacitances are in a ratio of approximately 1:2 as we would expect for the two different cell types. As shown in FIG. 4B, there is also a corresponding change in conductance that demonstrates the same 1:2 ration correlation between the cell types and conductance. These data demonstrate that the microfliudic device can be used for detecting cells and detecting differences in cellular DNA content.

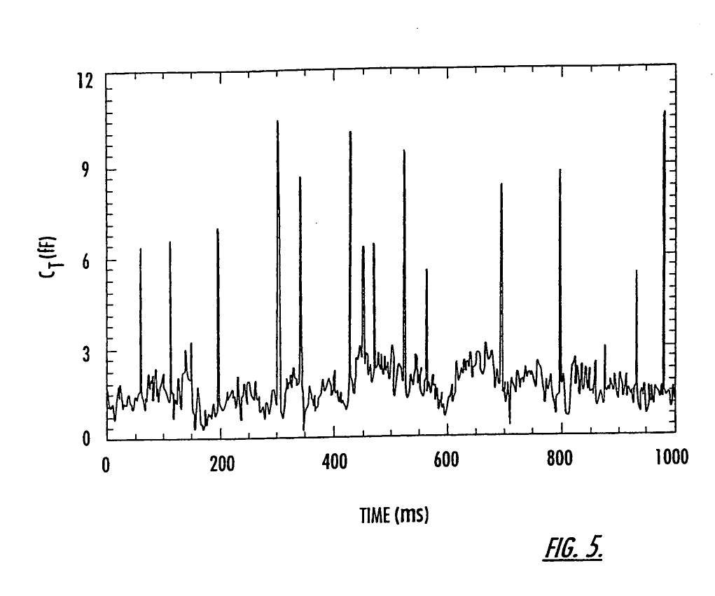

FIG. 5 shows the response of the microfluidic device over a course of 1000 ms to fixed mouse myeloma SP2/0 cells suspended in 75% ethanol and 25% phosphate buffered saline solution at 10° C. Distinct peaks are present in the data; each peak corresponds to a single cell flowing past the electrodes. The slight difference in peak widths is an artifact of the time-resolution limit of the data acquisition. The channel height of the device was 30 μm. As shown in FIG. 5, the response is a series of sharp peaks whose amplitudes ΔC_{T} range from ˜3 fF to ˜12 fF. The individual peaks are separated by time intervals ranging from 40-100 ms. Optical observations during similar measurement runs confirmed that each peak corresponded to a single cell flowing past the microelectrodes.

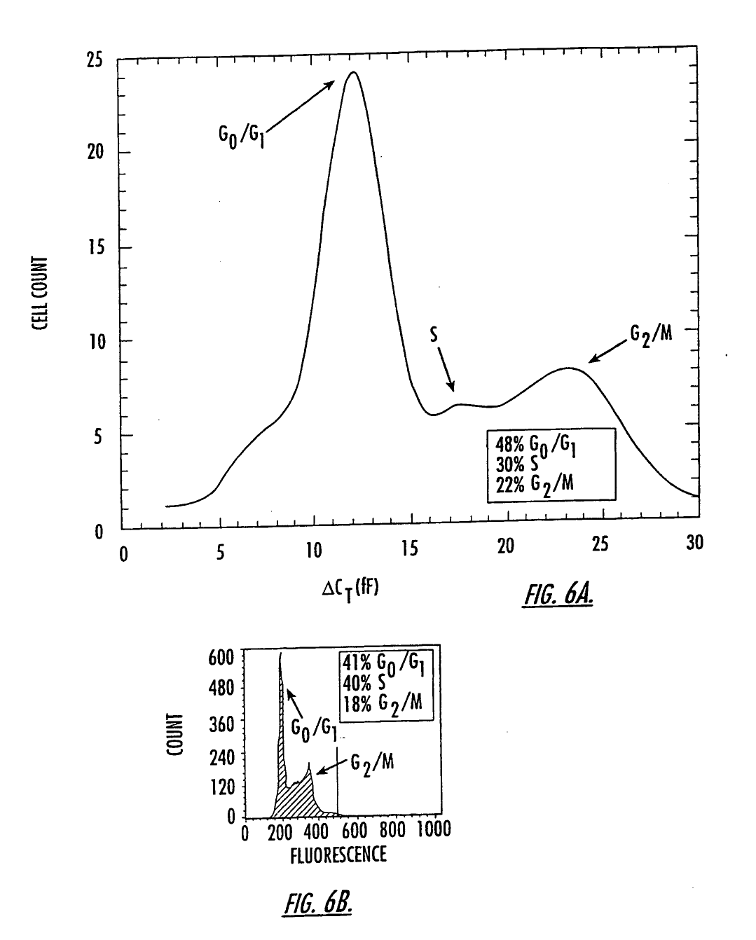

A central analysis technique in flow cytometry is the DNA histogram, which provides a visual representation of the number of cells as a function of DNA content and therefore the proportion of cells in each phase of the cell cycle. FIG. 6A is a histogram resulting from our capacitance measurements. FIG. 6A is the ungated histogram shows two major peaks, one centered at 12.3 fF, corresponding to G₀/G₁-phase, and one centered at 23.0 fF, corresponding to G₂/M-phase. The distribution of cells at capacitances less than 10 fF correspond to hypodiploid cells; the distribution of cells at capacitances greater than 27 fF are due to hyperdiploid cells. Based on the histogram obtained, we judged that approximately 48% are in G₀/G₁-phase, 30% in S phase, and 22% in G₂/M-phase. This cell cycle distribution is comparable to that obtained by conventional flow cytometry. FIG. 6B is a histogram obtained via conventional flow cytometry. The data has been gated and does not include hypo- and hyperdiploid cells. Two peaks at fluorescence channels 190 and 380 correspond to G₀/G₁- and G₂/M-phases, respectively.

As shown, there are two distinct populations of SP2/0 cells: one corresponding to 2N DNA content, centered at 12.3 fF and one corresponding to 4N DNA content, centered at 23.0 fF. Based upon this capacitance histogram, results show that approximately 48% of the cells are in G₀/G₁ phase, 30% S phase, 22% G₂/M phase, and <1% hyperdiploid. This distribution is comparable to that achieved with standard laser flow cytometry (FIG. 6B).

The histogram shown in FIG. 3A demonstrates that the capacitance cytometry device of the present invention is able to differentiate cells in different phases of the cell cycle. It is believed that the measured differences in capacitance are not due to cells flowing past the electrodes at different channel positions with respect to the electrodes since it has been optically confirmed that the cells flow in the center of the channel and directly between the electrodes. Since flow in the channel is laminar, it is not expected nor was it observed that lateral motion of cells occurs across the channel width. Over 60 devices have been tested and showed similar quantitative results, thus excluding irregularities of device fabrication.

### EXAMPLE 4

### Comparison of Avian Red Blood Cells

To experimentally confirm that cells are differentiated based on their DNA content and not by size or volume (G₀/G₁ cells have half the DNA content of G₂/M cells, and are also smaller), measurements and comparisons of avian red blood cells (Accurate Chemical and Scientific Corporation, Westbury, N.Y.) to mammalian (sheep) red blood cells (Sigma Chemical Company, St. Louis, Mo.), both fixed with glutaraldehyde were determined. Whereas avian red blood cells possess 2N DNA and are therefore in G₀/G₁-phase, mammalian red blood cells—the same 6-7 μm size as avian cells—contain no DNA.

Capacitance peaks were determined when avian cells flowed through device 10. No significant peaks were observed when interrogating the mammalian red blood cells—even after a series of experimental runs and measurements with a number of different devices, thereby confirming the measurement of DNA content rather than cell size or volume.

The avian red blood cells which were measured have an average capacitance change, ΔC_{T}, of 5.0 fF. Significant is the fact that avian red blood cells have less DNA content than SP2/0 cells and produce a smaller signal. Indeed, the ratio of observed signals of the two different types of cells (5.0 fF to 12.3 fF) is in remarkable quantitative agreement with the ratio of their DNA content (2.5 pg for Gallus domesticus versus 6.1 pg for Mus muscularis) (Tiersch, is T. R. & Wachtel, S. S. (1991) J. Hered. 82, 363-368; and Greillhuber, J., Volleth, M. & Loidl, J. (1983) J. Genet Cytol. 25, 554-560).

To determine the exact relationship between capacitance ΔC_{T} and DNA content, ΔC_{T} and DNA content were plotted for the different cell types measured. As shown, there is a linear relationship between ΔC_{T} and DNA content at 1 kHz frequency. Open circles correspond to data taken with a device whose channel height was 30 μm; open squares correspond to data taken with a device whose channel height was 40 μm. The 40 μm data were scaled by the ratio of the ΔC_{T}'s obtained for mouse SP2/0 cells measured with 30 μm- and 40 μm-high channel devices. All data were obtained at a temperature T=10° C. and in phosphate-buffered saline solution.

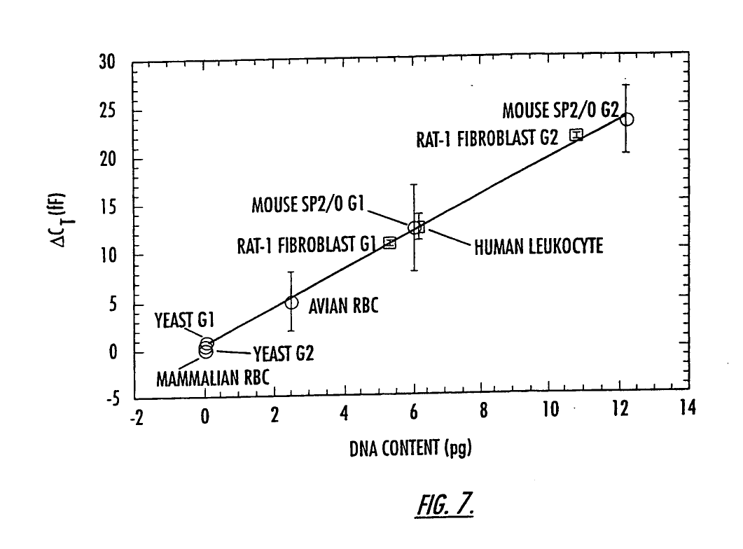

As shown in FIG. 7, there exists a strong linear dependence between the two at a frequency of 1 kHz. The ratio used to scale data taken with a 40 μm-high channel device was obtained by measuring mouse SP2/0 cells with both 30 μm- and 40 μm-high channel devices. The G₀/G₁ and G₂/M peaks were centered at 3.75 fF and 7.50 fF, respectively, when cells were measured with a 40 μm-high channel device; this in contrast to the G₀/G₁ and G₂/M peaks centered at 12.3 fF and 23.0 fF, respectively, when the same cells were measured with a 30 μm-high channel device.

These data demonstrate that there is a species-independent relationship between the DNA content of eukaryotic cells and the resulting change in capacitance as these cells transit in a low-frequency electric field. Since other cellular constituents may scale with DNA content (such as nuclear histones), it cannot be certain that the entirety of the capacitance signal is derived from DNA. However, the relationship between DNA content and ΔC_{T} holds across cells of the four species (yeast, mouse, rat, human) that were sampled. Since it is unlikely that all of these species have the same stoichiometric relationship between DNA and other nuclear and cytoplasmic constituents, the most likely explanation for the linear relationship between DNA content and capacitance signal is that the latter is strictly a function of the former.

### EXAMPLE 5

### Measurement of Progressive Alterations in DNA Content Using Flow Cytometry and Capacitance Cytometry

Capacitance cytometry was used to detect progressive alterations in DNA content. Rat-1 rodent fibroblast cells were synchronized in the G₀/G₁-phase of the cell cycle by placing them in serum-depleted media (containing 0.1% fetal bovine serum or FBS) for 72 hours. Subsequently, these cells were again permitted to grow in a serum-replete media (containing 10% FBS). Aliquots of cells were harvested from the depleted media and at intervals following the re-addition of serum. Cells were G₀/G₁-arrested (t=0 hr) and then allowed to progress through one mitotic cell cycle in synchrony. At t=12 hrs, the cells are beginning to enter S-phase and at t=21 hrs, they have fully entered this phase. At t=30 hrs, the cells have entered G₂/M-phase. This is shown by the secondary peak at ΔC_{T}=6.4 fF. At t=48 hrs, the cells have completed one mitotic cell cycle and are once again in G₀/G₁-phase. The G₀/G₁-, S-, and G₂/M-phases are indicated with arrows. The data shown were taken at T=10° C. with a microfluidic channel whose height was 40 μm. Measurement of DNA content was performed by both capacitance cytometry (using a 40 μm-high channel) and by standard laser flow cytometry.

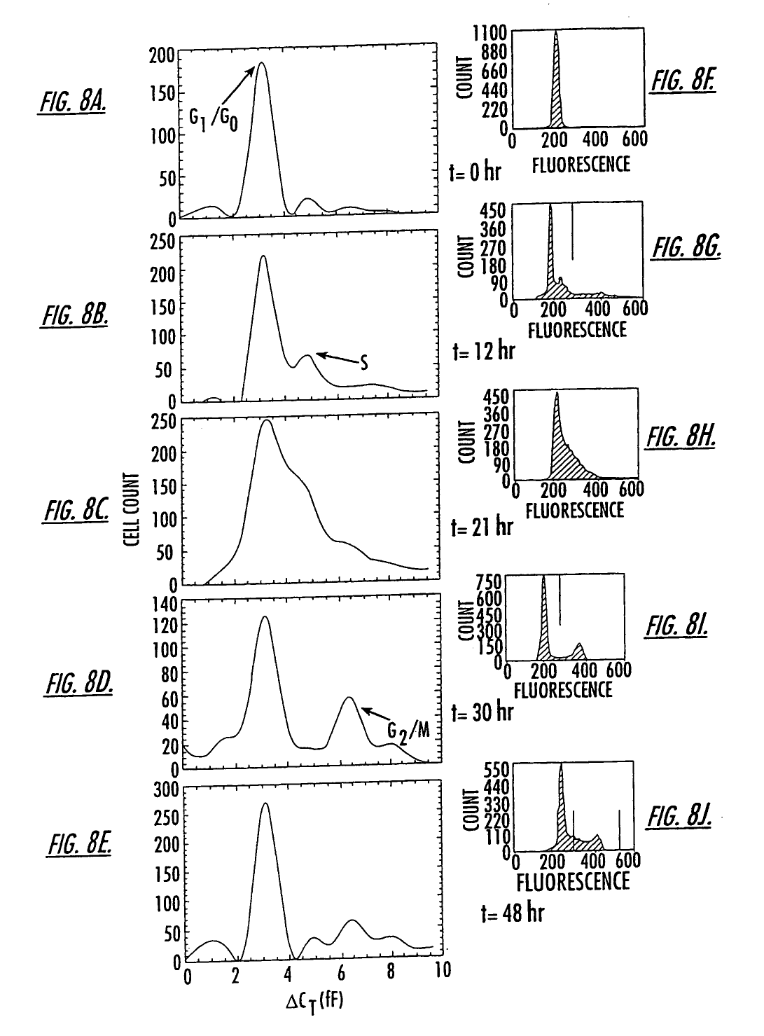

The results are shown in FIGS. 8A-J. A comparison of the histograms derived from capacitance cytometry and flow cytometry (FIGS. 8A-E capacitance cytometry; FIGS. 8F-J flow cytometry), indicated that cells cultured in the depleted media (t=0 hr) are synchronized at a single value of ΔC_{T} (centered at 3.2 fF in the histogram), which represents G₀/G₁-phase. Twelve hours following the addition of serum, the DNA content of some cells has increased as they enter the S-phase of the cell cycle. By 21 hours, most of the cells are in the S-phase, and contain an amount of DNA between G₀/G₁- and G₂/M-phase. By 30 hours, many of these cells have transited G₂/M-phase (centered at 6.4 fF), and by 48 hours, the cell population once more has the appearance of an asynchronously-growing population.

DNA content analysis is a core technique in examining cellular physiology. We have demonstrated that our integrated microfluidic device can replicate the DNA histograms of standard laser flow cytometry.

### EXAMPLE 6

### Detection of Single Molecules

The nanofluidic device of the invention is used to detect and measure the length of individual molecules. The individual molecules are pumped through the device by the input means, into the wide channel and flow through the narrow channel of the nanomicrofluidic device and pass between a series of nanoelectrodes. The changes in capacitance are measured using a capaticance network analyzer and impedence analyzer.

### EXAMPLE 7

### Detection of Labeled Polynucleotide Molecules

The microfluidic device of the invention was used to detect single molecules of DNA “treated” with different semiconductor nanocrystals whereby the nanocrystals attach themselves to the specific nucleotides. The different nanocrystals are then “read” using an on-chip capacitance electronic sensor, as the tagged DNA molecule flows past the sensor in a microfluidic channel. The sensor can detect and measure the delectric response of the nanocrystals. Using a different nanocrystal for each of the four nucleotides (A,T G, or C) there are only four different responses. Thereby, the DNA sequence is determined from the four different responses the microfluidic capacitance device detects as the differently labeled DNA molecules pass through the detector.

### EXAMPLE 8

### Detection of Liposomes and Measurement of Polynucleotide Content

DNA-filled liposomes have been created and can also be made of RNA and protein-filled liposomes. The change in capacitance of the DNA liposomes was measured using the microfluidic device of the invention and the results are shown in FIG. 15.

Liposomes are ideal because they can be made with ease, with each liposome having a relatively fixed quantity of DNA. Liposomes of up to 5 microns in diameter are made by first creating an inverted emulsion which encapsulates the solution of DNA using a single-chain alkane as the continuous phase, and the lipid as the surfactant. The emulsion is purified to produce monodisperse droplets using filtration. The droplets are then transferred from the alkane into a continuous aqueous phase, passing through an interface covered with a monolayer of a second lipid, whereupon they are coated with the second layer of the lipid bilayer. This then forms the stable liposomes.

Preliminary work indicates it is possible to create DNA-filled liposomes of approximately 5 μm in diameter. For this work, herring testis DNA (5 mg/mL) was employed, treated to remove RNA and most proteins, contained in an aqueous buffer. The resulting liposome contains approximately 33 pg of DNA. More homogeneous sources of DNA can be employed (phage or vector DNA, synthetic oligonucleotides in various conformations), which can be more robustly characterized with respect to purity, number of base pairs, and mass, as well as assure electrolyte concentrations that are appropriate to the cytosol of a typical human cell (for example, [K]=150 mM, [Na]=10 mM) [Ca]<100 nM). By performing capacitance measurements on this variety of DNA, it should be possible to characterize the way in which concentration, length, and GC content affect capacitance measurement.

### EXAMPLE 9

### Measurements of Filled Erythrocytes

The liposome membrane is much less complex than the membrane of typical human cell, and lacks ion channels and receptors that are likely to respond to polarizing electric fields and therefore affect the resulting capacitance signature. Consequently, DNA, proteins, lipids, and RNA, pre-inserted into mammalian erythrocytes, which normally contain no DNA but whose membranes have many of the electrical properties of other mammalian membranes. The approach will be to employ re-sealable white erythrocyte ghosts—erythrocytes that have been depleted of all of their cytosolic components. A variety of protein and nucleic acid molecules have been incorporated into erythrocytes using this technique, and it is possible to meticulously rid the erythrocyte membrane of its native cytosolic proteins prior to incorporation of the desired substance. The basic approach is to lyse erythrocytes in a hypotonic medium, add to the lysate the desired components (e.g., DNA), and then elevate the ionic strength to physiological levels. Finally, the cells are resealed by incubation at 37°. With this method, a substantial percentage of the cytosol (i.e., hemoglobin) remains within the erythrocyte. To obtain an erythrocyte from which virtually all of the cytosol is depleted, the method of Wood is employed. Washed erythrocytes are suspended in isotonic saline and permitted to flow (at 0-2° C.) onto an agarose column having an exclusion limit of 50 million daltons. The cells are lysed by washing in a hypotonic buffer. Since the effective path length for the released cytosol is three to four times that for the membranes, the membranes diffuse away from the cytosol. The membranes are collected, the appropriate electrolytes and macromolecules (i.e., proteins, DNA, RNA) added to the membranes, and the cells are resealed by incubation at 37° for 45 minutes. Although several precautions are necessary to prevent “leaky” membranes, the procedure is quite straightforward and has worked well in preliminary experiments in our laboratory.

Using the information gathered with the capacitance sensor, a dielectric shell model will be built to account for a number of different intracellular constituents (e.g., proteins and RNA), for the cellular membrane, and for the surrounding counter ions inside and outside the cell. There are a number of existing dielectric models of the cell, most notably that of Schwan; however, these models suggest that one should not detect DNA in the nucleus of cells, given the surrounding counter ions both in solution and in the cell and given the low frequency applied to the system. And yet there is a species-independent, linear relationship between the DNA content of eukaryotic cells and the change in capacitance. Clearly, a new model must account for this observation. The task set forth is challenging, as one will have to account for stray capacitances, ions present in both the buffer and the cell; however, it is not impossible, especially with the systematic approach outlined in these Aims. We will also be performing ab initio calculations, which, together, should provide a framework for a predictive, theoretical model. This model will be extended to account for the diverse dielectric responses of various cellular constituents (DNA, RNA, proteins, lipids) to a frequency sweep of the applied electric field from 0 to 10⁹ Hz.

### EXAMPLE 10

### Spectral Plots of DNA vs. Hemoglobin

The plots of changes in capacitance over a frequency range from 0 to 10⁹ Hz were generated comparing solutions of DNA and hemoglobin using the microfluidic device of the present invention. The data show that the capacitance changes as the frequency is varied.

It is understood that the above-described embodiments are illustrative of only a few of the many possible specific embodiments which can represent applications of the principles of the invention. Numerous and varied other arrangements can be readily derived in accordance with these principles by those skilled in the art without departing from the spirit and scope of the invention.

Various publications are cited herein, the disclosure of which are incorporated by reference in their entireties.

1. A microfluidic device comprising:
a substrate bearing a first electrode and a second electrode spaced a distance from said first electrode;
means for passing an individual particle or biological cell in the vicinity of said first and second electrodes;
means electrically connected to said first electrode for selectively applying an electrical signal;
means for detecting signals resulting from the application of the signal to said first electrode; and
means for determining a characteristic of said particle or biological cell from said detected signal.

2. The device of claim 1 wherein said means for passing an individual particle or biological cell in the vicinity of said first and second electrodes comprises:
fluid input means for inputting a fluid comprising a concentration of said particle or cell; and
a channel positioned adjacent said first electrode and said second electrode, said channel being coupled to said fluid input means, wherein said particle or biological cell flows through said channel.

3. The device of claim 2 wherein said channel has a width substantially the same as said distance of the spacing between said first electrode and said second electrode.

4. The device of claim 2 wherein said distance of the spacing of said first electrode and said second electrode and said width of said channel are selected such that in combination with a flow rate a said fluid from said fluid input means and said concentration of said biological cell in said fluid, said biological cells pass one by one through said channel between said first and second electrodes.

5. The device of claim 4 wherein said distance is in the range of 1 nm to 1 mm.

6. The device of claim 5 wherein said distance and said width of said channel are in the range of about 10 nm to about 50 μm.

7. The device of claim 6 wherein a height of said channel is in the range of about 10 nm to about 60 μm.

8-23. (canceled)

24. A microfluidic device comprising:
a substrate bearing a first electrode and a second electrode spaced a distance from said first electrode;
means for passing a fluid in the vicinity of said first and second electrodes;
means electrically connected to said first electrode for selectively applying an electrical signal;
means for detecting signals resulting from the application of the signal to said first electrode; and
means for determining from the detected signal a characteristic of said fluid.

25. The device of claim 24 wherein said means for passing a fluid in the vicinity of said first and second electrodes comprises:
fluid input means for inputting a fluid; and
a channel positioned adjacent said first electrode and said second electrode, said channel being coupled to said fluid input means, wherein said fluid flows through said channel.

26. The device of claim 24 wherein said channel has a width substantially the same as said distance of the spacing between said first electrode and said second electrode.

27. The device of claim 26 wherein said distance is in the range of 1 nm to 1 mm.

28. The device of claim 24 wherein said electrical signal applied to said first electrode is an AC voltage at a predetermined frequency.

29. The device of claim 24 wherein the applied frequency is between about 1 Hz and about 100 GHz.

30. The device of claim 29 wherein the applied frequency is in the range of about 1 kHz to about 100 GHz.

31. The device of claim 24 wherein said means for determining a characteristic of said cell comprises measuring impedance of said electrodes.

32. The device of claim 24 wherein said means for determining a characteristic of said cell comprises measuring change in total capacitance.

33-53. (canceled)

54. A method for measuring DNA content in a biological cell comprising:
providing a substrate bearing a first electrode and a second electrode spaced a distance from said first electrode;
passing an individual biological cell in the vicinity of said first and second electrodes;
applying an electrical signal to said first electrode;
detecting signals resulting from the application of the signal to said first electrode; and
determining a characteristic of said cell from said detected signal.

55. The method of claim 54 wherein said step of passing fluid in the vicinity of said first and second electrodes comprises:
fluid input means inputting a fluid; and
a channel positioned adjacent said first electrode and said second electrode, said channel being coupled to said fluid input means, wherein said fluid cell flows through said channel.

56. The method of claim 54 wherein said channel has a width substantially the same as said distance of the spacing between said first electrode and said second electrode.

57. The method of claim 54 wherein said distance and said width of said channel are in the range of about 10 nm to about 50 μm.

58-70. (canceled)
